(19) United States
(12) Patent Application Publication
(10) Pub. No.: US 2018/0064776 A1
(43) Pub. Date: Mar. 8, 2018

(54) COMPOSITION AND FORMULATION BASED ON PTERODON PUBESCENS BENTH. AND CORDIA VERBENACEA DC EXTRACTS/ESSENTIAL OILS, AND USES THEREOF

(71) Applicant: UNIVERSIDADE ESTADUAL DE CAMPINAS - UNICAMP, Campinas (BR)

(72) Inventors: Rosanna Tarkany Basting, Indaiatuba (BR); Humberto Moreira Spindola, Paulínia (BR); Leila Servat, Paulínia (BR); Nubia de Cassia Almeida Queiroz, Paulínia (BR); Ilza Maria Oliveira Sousa, Paulínia (BR); Rogério Grando, Paulínia (BR); Vanessa Helena da Silva Sousa, Paulínia (BR); Rodney Alexandre Ferreira Rodrigues, Paulínia (BR); João Ernesto De Carvalho, Paulínia (BR); Mary Ann Foglio, Paulínia (BR)

(21) Appl. No.: 15/543,182

(22) PCT Filed: Feb. 22, 2016
(86) PCT No.: PCT/BR2016/000018

§ 371 Date: Jul. 12, 2017

(30) Foreign Application Priority Data
May 28, 2015 (BR) BR10 2015 0124384

(51) Int. Cl. A61K 36/48; A61K 36/30; A61K 31/045; A61K 31/015; A61K 9/06; A61K 47/32; A61K 47/18; A61K 47/14; A61K 47/10; A61K 47/20; A61K 47/34; A61K 47/44

(52) U.S. Cl. CPC A61K 36/48; A61K 36/30; A61K 31/045; A61K 31/015; A61K 9/06; A61K 47/32; A61K 47/44; A61K 47/14; A61K 47/18; A61K 47/10; A61K 47/20; A61K 47/34; A61K 47/183

(57) ABSTRACT

The present invention relates to a composition based on a mixture of standard extracts/essential oils of Pterodon pubescens Benth. and Cordia verbenacea DC. Its use and formulations comprising said composition are additional objects. The composition has application in the pharmacological field, and more specifically, in the treatment of acute or chronic muscular-skeletal disorders associated with pain and/or inflammation, such as dislocations, edema, arthritis and trauma, among others. The composition provides maximum pharmacologic effect.

### FIELD OF THE INVENTION

The present invention relates to a composition based on a mixture of standard extracts/essential oils of Pterodon pubescens Benth. and Cordia verbenacea DC. Its use and formulations comprising said composition are additional objects.

The composition object of this invention has application in the pharmacological field, and more specifically, in the treatment of acute or chronic muscular-skeletal disorders associated with pain and/or inflammation, such as dislocations, edema, arthritis and trauma, among others. The composition. provides maximum pharmacologic effect.

### BACKGROUND OF THE INVENTION AND STATE OF THE ART

The use of medicinal plants is common in many communities where the population uses empirically plants for treating the symptoms of the disease, using teas and infusions. The union of popular knowledge and scientific research is called ethnopharmacology, defined by Rivier & Bruhn in 1979 as a “multidisciplinary area of research based on observation, description and experimental research of indigenous drugs and their biological activity”. The huge plant biodiversity is able to produce herbal medicines, phytopharmaceuticals and prototypes of new drugs economically relevant (Calixto, 2000; Newman & Cragg, 2007). In 2012, Newman & Cragg (2012) estimated that approximately 63% of all drugs were directly or indirectly derived from natural products (Newman & Cragg, 2012). Updated data in 2013 by the same authors increased that percentage to 75% (Newman & Cragg, 2013). Nature has been a source of medicinal agents for thousands of years and an impressive number of modern active substances has been isolated from natural sources, traditionally used for therapeutic purposes (Newman & Cragg, 2012). However, literature describes benefits of herbal medicines that are not observed in isolated compounds of the own species or in synthetic products (Heinrich et al., 2007).

Synergistic effects have been the key in phytotherapeutic research in recent years. Several studies have shown therapeutic superiority in combinations of different medicines, an effect that has not been shown in therapy with a single constituent. Plant extracts have several compounds that can synergistically interact by increasing the effectiveness and stability (Calixto, 2001). Literature describes benefits of herbal medicines that are not observed in isolated compounds of the own species or in synthetic products (Heinrich et al., 2007). It is believed that these therapeutic benefits found in herbal medicines are the result of synergistic interactions that increase their pharmacological effects, often with reduced side effects (Calixto, 2001). Example of this was the study described by Hildebert (2011) that demonstrated that standardized extracts of Ginkgo biloba at doses of 120 mg/Kg, containing 6 to 7 mg of total gincolides has effects equal to the administration of the mixture of the three components (gincolides A, B and C) isolated components in the same dose of 120 mg/Kg. The authors proposed that these results can be consequence of the flavonoids present in standardized extract that favors the absorption of active components. Therefore, these examples show that, in many situations, despite the discovery of the active ingredients, the mixture can provide better results (Heinrich et al., 2007; Hildebert, 2011).

The present invention relates to compositions based on the combination of crude extracts of Pterodon pubescens Benth. and the essential oil or extracts of Cordia verbenacea DC and their oral and topic uses. The composition of this invention comprises the extracts and standardized oils of Pterodon pubescens Benth. and of Cordia verbenacea DC, and the pharmaceutically acceptable vehicle. Moreover, the present invention also relates to formulations that, in addition to these main components, can also comprise optional components, such as emollient, wetting agent, thickener, chelant, antioxidant, preservative system, dyes and flavourings, and their use.

The genus Pterodon comprises four Brazilian native species: P. abruptus Benth., P. apparucuri Pedersoli, P. pubescens Benth., (P. emarginatus Vog,) and P. polygalaeflorus Benth. The plant species, Pterodon pubescens Benth., Botanical synonym Pterodon emarginatus Vog, is a tree of the Leguminosae-Papillonoideae family, popularly known in Brazil as faveiro, sucupira-branca, fava-de-sucupira, sucupira or sucupira-lisa, which can be mainly found in the savanna of the States of Minas Gerais, são Paulo, Goiás and Mato Grosso do Sul. An ethnobotanical study in 30 cities in the interior of Minas Gerais reported the popular use of sucupira, Pterodon pubescens Benth., in the treatment of rheumatism, sore throat and spine problems, such as tonic and depurative (Carvalho et al., 1999).

Preclinical studies have shown activity of the hydroalcoholic extract of seeds of P. pubescens in an experimental model of arthritis and the lack of sub-acute toxicity (Coelho et al. 2001). Some authors have suggested that the vouacapanic skeleton of furan diterpenes is involved with the anti-inflammatory properties of the oil from seeds of P. pubescens (Nunan et al., 1982; Carvalho et al., 1999; Silva et al., 2004, Spindola et al., 2009).

Silva et al. (2004) identified the compound 6α,7β-dihidroxivouacapane-17β-oic acid in the fraction that exhibited antiedematogenic activity in the paw edema assays induced by carrageenan or ear edena induced by croton oil, suggesting the effect of this compound in the anti-inflammatory activity of the fractions obtained from oil from seeds of P. pubescens.

Cordia verbenacea DC (Cv), a synonymous of Varronia verbenacea (DC.) Borhidi (Tropics, 2013), is a native plant of the Atlantic forest widely distributed along the southeastern Brazilian coast, belonging to the botanical family Boraginaceae, popularly known in Brazil as erva baleeira, maria milagrosa, catinga preta, baleeira-cambará, camaradinha, caraminha, caramoneira do brejo and maria-pretinha (Jornal da UNICAMP, 2005; Michielin et al., 2009). Several compounds are found in their aerial parts, including tannins, flavonoids, mucilage and also essential oils. These parts, leaves and inflorescences are used in popular medicine due to their antirheumatic, anti-inflammatory, antimicrobial and analgesic effect, and for their healing properties, in the form of alcoholic extract, decoction and infusion (Sertié et al., 2005; De-Carvalho et al., 2004; Passos et al., 2006).

The first studies of the anti-inflammatory properties of C. verbenacea bagen in the 80's by Sertié et al. (1988, 1990, 1991) which identified the artemethine compound in the extracts evaluated in pharmacological models, attributing the anti-inflammatory activity to this flavonoid. However, Bayeux et al. (2002), evaluated the antiedematogenic activity of the dichloromethane crude extract and of the fraction enriched with artemethine in the model of carrageenan-induced paw edema. The extract inhibited the edema, but the fraction of artemethine showed no activity. The dichloromethane extract also presented antinociceptive activity. Passos et al., (2006), demonstrated the presence of multiple components through the phytochemical analysis of ethanol extract, such as tannins, flavonoids, mucilage and essential oil. The main constituents of the essential oil of Cordia verbenacea DC identified by gas chromatography/mass spectrometry were α-humulene (29.69%), trans-caryophyllene (25.27%) and allo-aromadendrene (9.99%). Villa et al., in 2009, also analyzed the essential oil of Cordia verbenacea: more than 91% (46 constituents) have been identified. The oil evaluated was mainly composed of terpenic hydrocarbons, monoterpenes (41.5%) and sesquiterpenes (42.7%). The major identified compounds were α-pinene (36.5%), β-caryophyllene (11.7%) and α-santalene (8.6%). It also presented significant amounts of allo-aromadendrene (4.3%) and α-humulene (3%) (Vila et al., 2009). Due to the diversity of chemical groups found in extracts of this plant, there are several pharmacological activities reported for this species: antibacterial, anti-inflammatory, antinociceptive, antiedematogenic, antiulcerogenic, antitumor, antifungal, anticancer and antimicrobial, among others (De carvalho et al., 2004; Passos et al., 2006; Fernandes et al., 2007; Sertié et al., 2005; Roldão et al., 2008; Kviecinski et al., 2010; Parisotto et al., 2012; Rodrigues et al., 2012; Matias et al., 2010; Schlemper et al., 2000). According to Michielin (2009), Cordia verbenacea contains anti-inflammatory substances whose efficacy and reduced side effects that places it as a safe and effective alternative in the topical treatment of inflammations.

As it has been described, both species have anti-inflammatory and antinociceptive activity confirmed. The great advantage of this invention is the use of two standardized extracts/oil in combination, where it has been experimentally observed that the mixture between them extends the pharmacological activity, acting in synergy for the treatment of chronic inflammatory diseases by oral and topical use, and with the consequent reduction of the effective dose used of each plant species.

On the concern of developing a trade composition, it is highlighted that this application meets the process as a whole, from the standardization of extracts, essential for effectiveness, since it meets specifications of minimum amounts of active compounds, through validation of analytical methodology for evaluation of compounds with pharmacological activity and the preparation of formulations designed according to proposed applications, essential to the success of the final application.

### BRIEF DESCRIPTION OF THE INVENTION

The present invention relates to a composition based on a mixture of standard extracts/essential oils of Pterodon pubescens Benth. and Cordia verbenacea DC. Its use and formulations comprising said composition are additional objects.

The composition comprises the following components:
Standardized extract of Pterodon pubescens Benth.;
Standardized extract of Cordia verbenacea DC; and
pharmaceutically acceptable vehicle.

The standardized extract of fruits and seeds of Pterodon pubescens Benth. Comprises variable concentration between 0.2% and 5%, preferably 2.5% of Cordia verbenacea DC with concentrations ranging between 0.2% and 5%, and preferably 2.5%.

The extract of Pterodon pubescens Benth.; Comprises the compounds geranylgeraniol and vouacapanes in varying concentrations between 15 and 40% and Cordia verbenacea DC extract, comprising the compound α-humulene and trans-caryophyllene in variable concentrations between 1.0% to 20%.

The preferred adjuvant is selected among water, saline or buffer solutions in a percentage adequate to (amount sufficient to) achieve 100% of the formula based on the total weight of the composition.

The composition comprises applying on the preparation of formulations for treating acute or chronic muscular-skeletal disorders associated with pain and/or inflammation, such as dislocations, edema, arthritis and trauma.

The standardized extract of Pterodon pubescens Benth. And of Cordia verbenacea DC is used in the preparation of compositions for treating acute or chronic muscular-skeletal disorders associated with pain and/or inflammation, such as dislocations, edema, arthritis and trauma.

The formulations comprise the composition described and optional components selected from chelating agents, pH adjusters, preservative agents, emollients, humectants, thickener, bacteriostatic agents, active ingredients, colorants and flavourings.

### BRIEF DESCRIPTION OF THE DRAWINGS

FIG. 1 presents the chemical structure of the compounds geranylgeraniol (m/z 288), 6α,7β-dihydroxyvouacapane-17β-oate methyl ester (m/z 362) and the ester isomers 6α-hydroxy-7β-acetoxy-β-vouacapane-17β-oate methyl ester (m/z 404 A) and 6α-acetoxy-7β-hydroxy-vouacapane-17β-oate methyl ester (m/z 404 B).

FIG. 2 presents the chemical structure of compounds α-humulene and trans-caryophyllene.

FIG. 3 refers to weight changes of the animals treated with the combination of extracts and the vehicle.

FIG. 4 refers to the results obtained between the combination of extracts on abdominal contortion model by acetic acid.

FIG. 5 refers to the overall effect of the two compounds when compared with the summation of the effects of the separated compounds.

FIG. 6 refers to the result expressed by the difference in volume (mL) displaced between the measured paws.

FIG. 7 refers to percentage of inhibition compared to the vehicle in different times of evaluation.

FIGS. 8 and 9 refer to the data and the percentage of increase in tolerance to mechanical stimuli in day 1 and day 7, respectively, after administration of the composition only on day 1 and 7.

FIGS. 10 and 11 refer to the data and the percentage of increase in tolerance to mechanical stimuli in day 1 and day 7, respectively, after daily administration of the composition.

### DETAILED DESCRIPTION OF THE INVENTION

The present invention relates to a composition based on a mixture of standard extracts/essential oils of Pterodon pubescens Benth. and Cordia verbenacea DC. Its use and formulations comprising said composition are additional objects.

The composition comprises standardized extract of fruits and seeds of Pterodon pubescens Benth. With concentration varying between 0.2% and 5%, preferably 2.5% of Cordia verbenacea DC with concentrations ranging between 0.2% and 5%, and preferably 2.5%.

The extract of Pterodon pubescens Benth.; comprises the compounds geranylgeraniol and vouacapanes in concentrations varying between 15 and 40% and Cordia verbenacea DC extract, comprising the compound α-humulene and trans-caryophyllene in concentrations varying from 1.0% to 20%.

The preferred adjuvant is selected among water, saline or buffer solutions in a percentage adequate to (amount sufficient to) achieve 100% of the formula based on the total weight of the composition.

The pharmaceutical compositions of this invention are described below, but they are not limited to the extract/oil of Pterodon pubescens and Cordia verbenacea ratios.

### Carbopol® Gel

The amounts of the components used for preparing Carbopol gel are described in table 1.

TABLE 1
Composition of 100 g gel formulation of Carbopol 940 ®

| | Formulation | Amounts |
|---|---|---|
| Component | function | (g) |
| Carbopol 940 ® | Thickener | 3.00 |
| EDTA | Chelant | 0.10 |
| Methylparaben | Preservative | 0.20 |
| Propylparaben | Preservative | 0.10 |
| Triethanolamine | Alkalizing agent | 1.00 |
| Glycerin | Wetting agent | 5.00 |
| Sodium lauryl sulfate* | Surfactant | 1.00 |
| Extract/Essential oil (g) | Active | 2.50 |
| H₂O (qsp) | Solvent | 100 |

*Add last in the formulation

Carbopol®, preservatives and EDTA were added to distilled water and EDTA (ethylenediaminetetraacetic acid), and was left to stand for 24 hours. After then, manual shaking began with a polyethylene spatula for 15 minutes, adding the other components, and lastly, sodium lauryl sulfate. The active principles were dispersed in glycerine for better incorporation into the gel.

### Aristoflex® Gel

The amounts of the components used for preparing Aristoflex® gel are described in table 2.

TABLE 2
Composition of Aristoflex ® gel formulation

| | Amounts | |
|---|---|---|
| | Formulation | Formulation |
| Component | (g) | function |
| Aristoflex AVC ® | 5.00 | Thickener |
| Methylparaben | 0.10 | Preservative |
| Propylparaben | 0.10 | Preservative |
| EDTA | 0.10 | Chelant |
| Propylene glycol | 3.00 | Wetting agent |
| Sodium lauryl sulfate* | 1.00 | Surfactant |
| Extract/Essential oil | 2.50 | Active |
| H2O qsq | 100 | Solvent |

*Add last in the formulation

Aristoflex®, preservatives and EDTA have been added to distilled water. It was continuously stirred with a glass rod until complete dissolution. Then, the active principle was incorporated, and lastly, sodium lauryl sulfate.

### Nanoemulsion

The amounts of the components used for preparing the nanoemulsion are described in table 3.

TABLE 3
Nanoemulsion composition

| | |
|---|---|
| | Amounts |
| Component | (g) |

Oily phase

| | |
|---|---|
| Ethyl acetate P.A. | 10 |
| Pluronic | 0.2 |
| Polyacrylamide | 0.2 |
| Active principle | 0.2 |
| (extract/oil) | |
| Vegetable oil | 0.5 |
| Tween 327 | 0.1 |

Aqueous Phase

| | |
|---|---|
| Distilled water | 15 |
| Lauryl sulphate | 0.05 |
| Tween 327 | 0.05 |

The phases were prepared separately under magnetic stirring for 30 minutes. After complete dissolution of the components, the oily phase was added to the aqueous phase by dripping, under mechanical stirring (Ultra-Turrax®). After complete incorporation of the phases, the ethyl acetate was evaporated in a rotary evaporation system under vacuum.

The composition described in this invention can be prepared using any known state of the art process. Oil from the seeds or fruits of P. pubescens can be extracted by pressing or employing low-polarity (hexane), medium polarity (dichloromethane or ethyl acetate) and high polarity (ethanol or water) solvents, alone or in combination, and also by a steam-dragging hydrodistillation process for obtaining the essential oil. The ratio of plant material:solvent can vary between 1:1 to 1:10, and is preferably 1:5. Then, the solution obtained is concentrated under vacuum to dryness.

The essential oil of Cordia verbenacea DC can be extracted from fresh leaves and smudged through the steam-dragging hydrodistillation process, which is carried out for a period of 1.5 to 2 hours. At the end of the process, the oil is separated from water in a separating funnel, followed by using anhydrous sodium sulphate P.A. to eliminate residual water amount. The oil can be stored in a freezer up to its use. Extracts from fresh leaves of Cordia can be also prepared using isopropanol, ethyl acetate or hydroalcoholic mixtures.

To allow a better understanding of the present invention and clearly demonstrate the technical advances achieved, the results obtained are presented herein.

Standardized Extract of Pterodon pubescens Benth

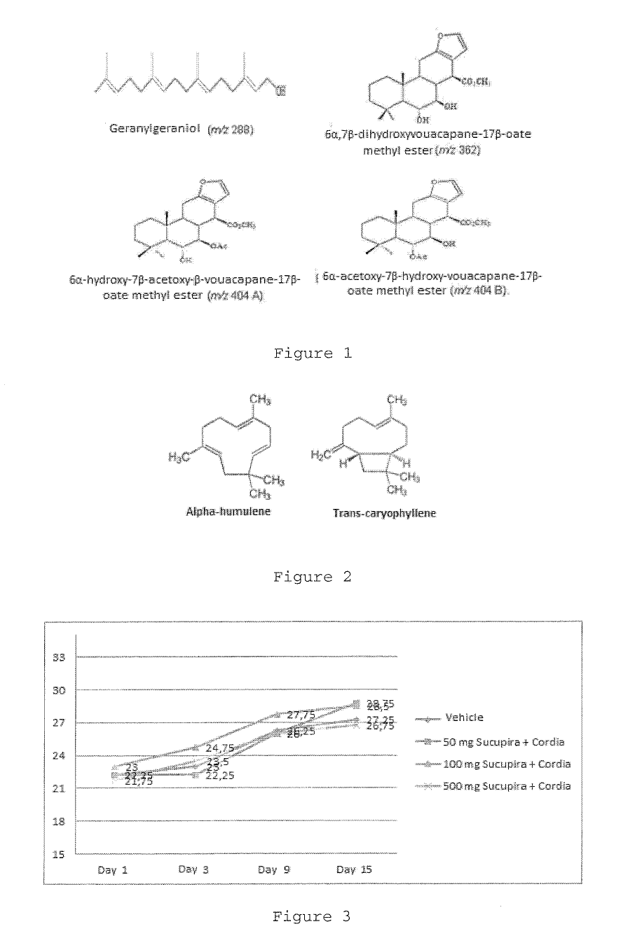

The extract must be produced with the fruits and seeds of Pterodon pubescens, being used a liquid extractant of high, medium or low polarity, and the concentration of the compounds geranylgeraniol and vouacapanes (FIG. 1) should be 15 to 40%.

Standardized Extract of Cordia verbenacea DC

The essential oil should be produced with fresh leaves of Cordia verbenacea DC by steam-dragging distillation process, and the concentration of markers α-humulene and trans-caryophyllene should be 1.0% to 20%. FIG. 2 presents the chemical structure of compounds α-humulene and trans-caryophyllene (FIG. 2).

Different animal models were used to evaluate the antinociceptive and anti-inflammatory activity profile.

### Single Dose Toxicity Study

Female Swiss mice have been used in the acute toxicity and hippocratic screening test, divided into groups (n=6): one group was treated with vehicle (saline) and the other were treated with an acute dose of 50, 100 and 500 mg/Kg of the mixture of dichloromethane extract of seeds of Pterodon pubescens (Pp) and the essential oil of Cordia verbenacea (Cv) at a rate of 1:1. The initial dose selected is the one most likely to cause mortality, according to normative reference No. 420 of OECD, 2002—Organization for Economic Cooperation and Development). The treatments were conducted orally through a flexible gastric probe and, after administration of the mixture, systematic behavioral observations were carried out to assess the hippocratic screening, which provides a general estimate of the toxicity of the substance on the conscious state and general disposition, activity and coordination of the motor system, reflections and activities on the central nervous system and the autonomic nervous system. Parameters (such as: general activity, vocal tremor, irritability, touch response, response to tail clamping, contortion, rear train position, straightening reflex, body tonus, strength to grab, ataxia, hearing reflex, corneal reflex, tremor, seizure, straub, hypnosis, anesthesia, lacrimation, ptosis, urination, defecation, piloerection, hypothermia, breathing, cyanosis, hyperemia, death) were assessed in 15 min, 30 min, 1 h, 2 h, 4 h and 8 h after administration and, daily, until the fourteenth day (Britto, 1994). On the fourteenth day, the animals were anesthetized and, thereafter, they were sacrificed after macroscopic analysis of the organs (heart, lungs, spleen, kidneys, ovaries and uterus) and they were weighted to determine their relative weights (body weight by 100 g of body weight).

This assay demonstrated the lack of signs of toxicity of the combination of extracts, evidenced by the normal progress of animal weight treated with the combination (FIG. 3), by the lack of alterations in the treated animals during evaluation of the hippocratic screening and the macroscopic analysis of the organs.

### Antinociceptive and Anti-Inflammatory Activity Evaluation and Confirmation of the Synergistic Effect of the Combination.

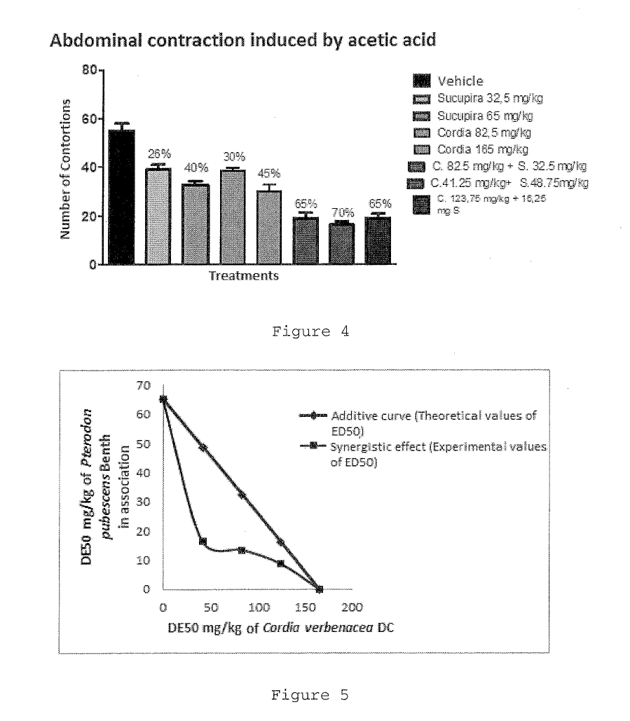

In the models for evaluating the antinociceptive activity, tests were carried out with extracts separately for determining the effective dose (DE50) of each extract in the abdominal contortion induced by acetic acid. For the extract of Pterodon pubescens, the effective dose was 65 mg/Kg and for the essential oil of Cordia verbenacea it was 165 mg/Kg. After determining the effective dose of each extract, test were carried out with associations of extracts at ratios that varied from 200% to 0%, with 5% variation range of the effective dose of each extract in each mixture. The best results found with the combination of extracts on the abdominal contortion model by acetic acid are shown in FIG. 4.

For proving the actual synergistic effect, an isobologram was used, which is a mathematical model that allows the envision of three types of pharmacological interactions, which are synergism, addition and antagonism. First, in the construction of isobologram, the DE50 of each extract experimentally defined were inserted separately in the abscissa and ordinate axis, and later joined, generating an additive isobole. Subsequently, the data experimentally obtained in the abdominal contortion model induced by acetic acid of the combination of extracts were analyzed in relation to their positions before the additive isobole. If the points are positioned around the additive isobole, the action will be of additivity; if they are positioned below, the action will be considered as synergism and, if they are positioned above, the action will be considered antagonism (Berembaum, 1989). As it is demonstrated in FIG. 5, the overall effect of the two compounds is better than the summation of the effects of the compounds separated, demonstrating a real synergism of extracts.

### Antiedematogenic Activity Assessment

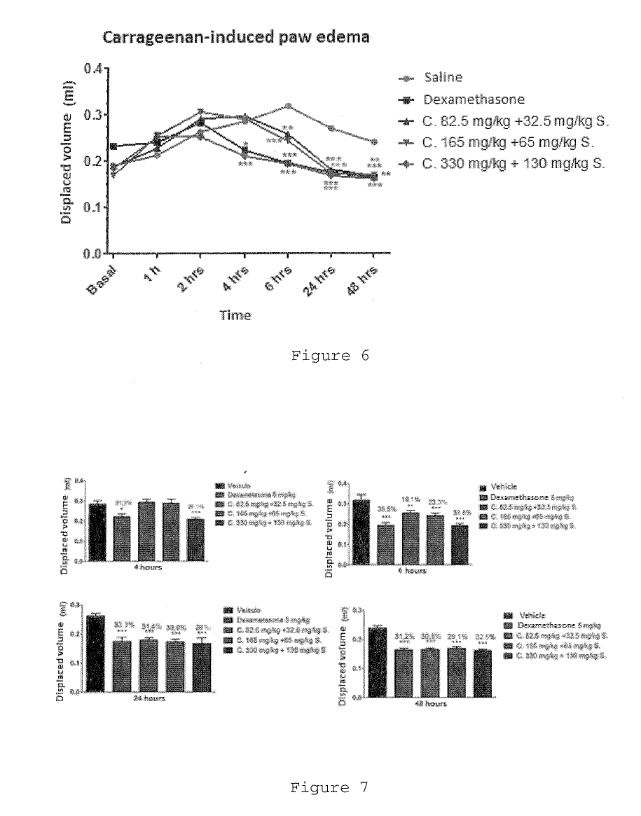

In order to verify the possible antiedematogenic activity of the mixture of dichloromethane crude extract of Pterodon pubescens and essential oil of Cordia verbenacea, the method of inducing paw edema with carrageenan in Swiss mice was used (Winter et al., 1962). The fasting animals (n=6) were divided into groups: negative control (salina), positive control (dexamethasone 5 mg/Kg) and the mixture of Pp+Cv in concentrations of: 1) 82.5 mg/kg of Cordia+32.5 mg/kg of Pterodon, corresponding to 50% of the effective dose of each extract; 2) 165 mg/kg of Cordia+65 mg/kg of Pterodon, corresponding to 100% of the effective dose of each extract and 3) 330 mg/kg of Cordia+130 mg/kg of Pterodon, corresponding to 200% of the effective dose of each extract. The treatments (mixture and control groups) were orally administered 1 hour before the induction of edema by subcutaneous injection of 20 μL of carrageenan/paw (1%) in the subplantar region of the right hind paw. The inflammation was assessed by measuring the paw volume for 4 hours, every hour, after injecting 20 μL of carrageenan using a digital plethysmometer (UGO-Basile). The result was expressed by the difference in volume (mL) displaced between the measured paws (FIG. 6). The percentage of inhibition compared to the vehicle in different times of evaluation is expressed in FIG. 7.

### Evaluation of Anti-Inflammatory Activity—Comparison Between Acute and Chronic Treatment—CFA-Induced Allodynia (Freund's Adjuvant)

The procedures carried out were similar to the method described by Villetti (2003). A solution of CFA (Complete Freund's Adjuvant) was used to induce inflammation. Experimental groups of 6 male Swiss mice received CFA on the right paw in a constant volume of 40 μL (s.pl.). A low pressure mechanical stimulus (0-8 g for a period of 5 seconds), corresponding to the painful response to the touch (allodynia) was produced with the aid of a Dynamic Plant Anaestesiomether apparatus (Ugo Basile, model 37450). Three consecutive evaluations were carried out in the right paws, and the averages were used for calculating the allodynia indexes. The basal index was measured before the CFA application on day 0, and the animals considered ideal for being subjected to testing were those which produced a mean value around 8. After CFA injection, the measurements were carried out considering 2 distinct phases: 24 h on day 1 (allodynia response to subacute pain); and day 7 (allodynia response to chronic pain). Vehicle (10 mL/Kg—negative control), dexamethasone (5 mg/Kg—positive control) and the mixture of Pp+Cv in concentrations of: 1) 82.5 mg/kg of Cordia+32.5 mg/kg of Pterodon, corresponding to 50% of the effective dose of each extract; 2) 165 mg/kg of Cordia+65 mg/kg of Pterodon, corresponding to 100% of the effective dose of each extract and 3) 330 mg/kg of Cordia+130 mg/kg of Pterodon, corresponding to 200% of the effective dose of each extract. The assessment of the anti-allodynia activity was carried out after 30, 60 and 90 minutes of oral administration of the extracts and the positive and negative controls, on days 1 and 7 of the assay.

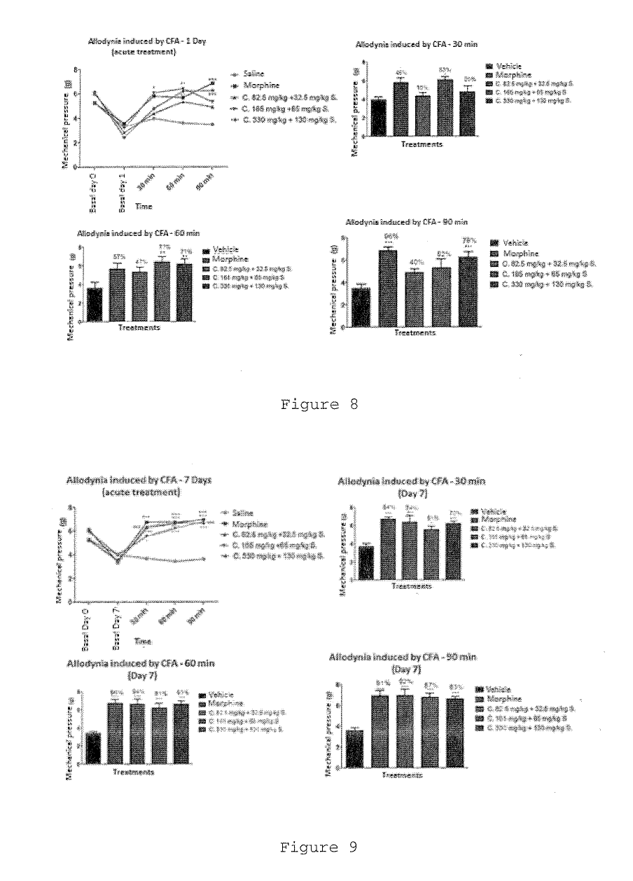

In a first assay, the administration of the combination of extracts only on days of assessment of mechanical allodynia was carried out, on day 1 (FIG. 8) and on day 7 (FIG. 9), 30 minutes before the evaluation of the anti-allodynia effect.

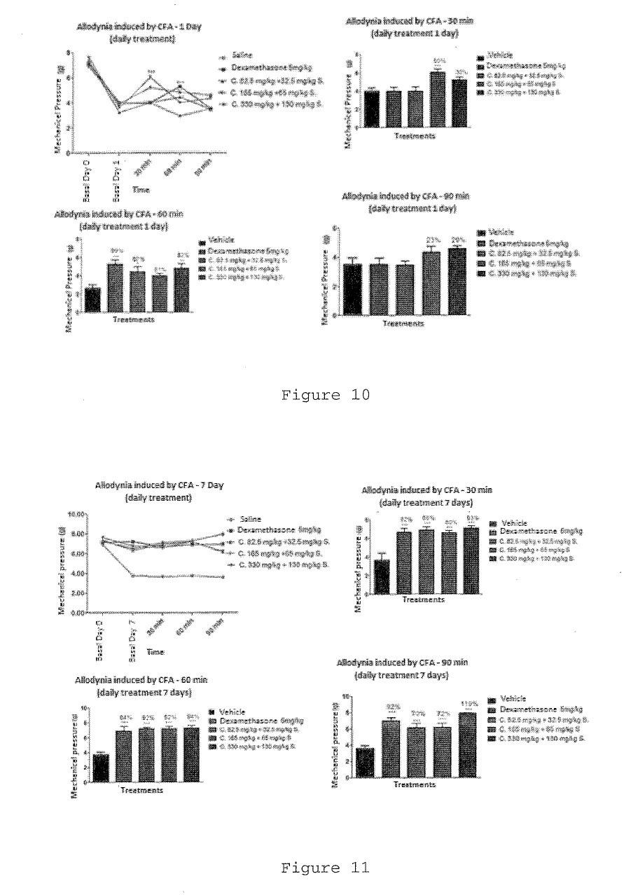

In another assay, the daily administration of the extract combination was carried out, being on day 1 (FIG. 10) and on day 7 (FIG. 11), 30 minutes before the evaluation of the anti-allodynia effect. FIGS. 10 and 11 contain the data and the percentage of tolerance increase to mechanical stimuli on day 1 and day 7, respectively.

It was possible to verify, with the daily treatment of the extracts combination that there is an extension of the pharmacological effect and anti-inflammatory activity, with a consequent increase in the level of tolerance to mechanical touch and increased basal level when compared to the groups treated only on days 1 and 7 of the anti-allodynia assessing.

According to the results obtained in preclinical studies, it was verified that the extracts of Pterodon pubescens associated with the essential oil/extracts of Cordia verbenacea feature a potential anti-inflammatory and antinociceptive activity, demonstrating real synergistic effect, showing better pharmacological effects, extension of the pharmacological activity and lower effective doses of each extract when used in combinations.

For preparing the pharmaceutical formulations suggested, there may be used different ratios of the mixture of Pterodon pubescens extracts and essential oil/extracts of Cordia verbenacea, varying from the ratio of 0.1 to 99.9% of each extract in the combination. Additionally to the concentration of active principles, the other components used in the formulations can vary as well. A description of some pharmaceutical forms using the combination of plant species is hereinbelow.

1. A composition comprising the following components:
a standardized extract of Pterodon pubescens Benth.;
a standardized extract of Cordia verbenacea DC; and
a pharmaceutically acceptable vehicle.

2. The composition according to claim 1, wherein the standardized extract of Pterodon pubescens Benth comprises a fruit and seed extract, having a concentration varying between 0.2% and 5%, preferably 2.5%, and the standardized extract of Cordia verbenacea DC having a concentration ranging between 0.2% and 5%, preferably 2.5%.

3. The composition according to claim 2, wherein the standardized extract of Pterodon pubescens Benth comprises the compounds geranylgeraniol and vouacapanes in concentrations of between 15 and 40%; and the standardized extract of Cordia verbenacea DC comprises the compounds α-humulene and trans-caryophyllene in concentrations of from 1.0% to 20%.

4. The composition according to claim 1, wherein the pharmaceutically acceptable carrier is selected from the group consisting of water, saline and buffer solutions in an amount sufficient to achieve 100% of the formula based on the total weight of the composition.

5. A method for treating an individual suffering from acute or chronic muscular-skeletal disorders associated with pain and/or inflammation, which comprises administering to the individual in need of treatment, the composition according to claim 1.

6. The method of claim 5, wherein the acute or chronic muscular-skeletal disorders associated with pain and/or inflammation are from dislocations, edema, arthritis and trauma.

7. The composition according to claim 1, and further comprising one or more optional components selected from the group consisting of chelating agents, pH adjusters, preservative agents, emollients, humectants, thickener, bacteriostatic agents, active ingredients, colorants and flavourings.

8. The composition according to claim 7, in the form of a gel, and comprising 3.00 grams of a crosslinked polyacrylic acid polymer; 0.10 gram of EDTA; 0.20 gram of methylparaben; 0.10 gram of propyl paraben; 1.00 g of triethanolamine; 5.00 grams of glycerin; 1.00 gram of sodium lauryl sulfate; 2.50 grams of Pterodon pubescens Benth. and Cordia verbenacea DC extract and a sufficient amount of water to make up 100 grams.

9. The composition according to claim 7, comprising 5.00 grams of ammonium acryloyldimethyltaurate/VP copolymer; 0.10 gram of methylparaben; 0.10 gram of propylparaben; 0.10 gram of EDTA; 3.00 grams of propyleneglycol; 1.00 gram of sodium lauryl sulfate*; 2.50 grams of Pterodon pubescens Benth. and Cordia verbenacea DC extract and a sufficient amount of water to make up 100 grams.

10. The composition of claim 7, wherein the oily phase comprises 10 grams of ethyl acetate P.A.; 0.2 gram of Pluronic; 0.2 gram of polyacrylamide; 0.2 gram of Pterodon pubescens Benth and Cordia verbenacea DC extract.; 0.5 gram of vegetable oil and 0.1 gram of Tween 327; and the aqueous phase comprises 15 grams of distilled water; 0.05 gram of sodium lauryl sulphate and 0.05 gram of Tween 327.

11. A method for treating an individual suffering from acute or chronic muscular-skeletal disorders associated with pain and/or inflammation, such as dislocations, edema, arthritis and trauma, which comprises administering to said individual in need of treatment, the composition according to claim 7.

12. A method for treating an individual suffering from acute or chronic muscular-skeletal disorders associated with pain and/or inflammation, such as dislocations, edema, arthritis and trauma, which comprises administering to said individual in need of treatment, the composition according to claim 2.

13. A method for treating an individual suffering from acute or chronic muscular-skeletal disorders associated with pain and/or inflammation, such as dislocations, edema, arthritis and trauma, which comprises administering to said individual in need of treatment, the composition according to claim 3.

14. A method for treating an individual suffering from acute or chronic muscular-skeletal disorders associated with pain and/or inflammation, such as dislocations, edema, arthritis and trauma, which comprises administering to said individual in need of treatment, the composition according to claim 4.

15. The composition according to claim 2, and optionally including one or more components selected from chelating agents, pH adjusters, preservative agents, emollients, humectants, thickener, bacteriostatic agents, active ingredients, colorants and flavourings.

16. The composition according to claim 3, and optionally including one or more components selected from chelating agents, pH adjusters, preservative agents, emollients, humectants, thickener, bacteriostatic agents, active ingredients, colorants and flavourings.

17. The composition according to claim 4, and optionally including one or more components selected from chelating agents, pH adjusters, preservative agents, emollients, humectants, thickener, bacteriostatic agents, active ingredients, colorants and flavourings.

18. A method for treating an individual suffering from acute or chronic muscular-skeletal disorders associated with pain and/or inflammation, such as dislocations, edema, arthritis and trauma, which comprises administering to said individual in need of treatment, the composition according to claim 8.

19. A method for treating an individual suffering from acute or chronic muscular-skeletal disorders associated with pain and/or inflammation, such as dislocations, edema, arthritis and trauma; which comprises administering to said individual in need of treatment, the composition according to claim 9.

20. A method for treating an individual suffering from acute or chronic muscular-skeletal disorders associated with pain and/or inflammation, such as dislocations, edema, arthritis and trauma, which comprises administering to said individual in need of treatment, the composition according to claim 10.
